(19) United States
(12) Patent Application Publication
(10) Pub. No.: US 2007/0226882 A1
(43) Pub. Date: Oct. 4, 2007

(54) Sports eye protector

(76) Inventor: Elizabeth A. Ryan, Florence, MA (US)

Correspondence Address: Elizabeth A. Ryan, 711 Park Hill Road, Florence, MA, 01062, US

(21) Appl. No.: 11/729,227

(22) Filed: Mar. 28, 2007

Related U.S. Application Data
(60) Provisional application No. 60/743,839, filed on Mar. 28, 2006.

(51) Int. Cl. A61F 9/02

(52) U.S. Cl. 2/426

(57) ABSTRACT

In one aspect, there is a sports eye protector that includes a frame, a padding in communication with the frame and a sports grille releasably attachable to the frame. In another aspect, there is a sports eye protector that includes a frame and a sports grille releasably attachable to the frame, wherein the sports grille is compliant with a testing standard associated with a sport. In yet another aspect, there is a sports eye protector that includes a frame configured to be releasably attachable to any one of a plurality of sports grilles, wherein the plurality includes a first sports grille associated with a first sport and a second sports grille associated with a second sport.

### CROSS-REFERENCE TO RELATED APPLICATIONS

This application claims priority to and the benefit of, and incorporates herein by reference in its entirety, U.S. Provisional Patent Application No. 60/743,839, filed on Mar. 28, 2006, entitled “Sports Eye Protector.”

### TECHNICAL FIELD

This invention relates to eye protection, specifically to eye protection for sporting activities.

### BACKGROUND

Eye protectors in certain sports are required, and in other sports, are voluntarily used by the wearer. Eye protectors that are used in certain sports may have testing criteria established by a supervising body such as the American Society for Testing and Materials (ASTM). Testing criteria for eye protectors can involve testing to determine how objects may interact with the eye protector, such objects include: balls, pucks, sticks, racquets, and body parts, such as a finger. Testing criteria may certify a product for a particular sport, for a particular sport at a certain age group, and/or for more than one sport. For example, in testing eye protectors, baseball has four levels of testing speeds for four age levels. In another example, eye protectors that pass testing criteria for racquetball also pass testing criteria for paddleball. However, other sports, like ice hockey and paint ball each have their own standards and the testing is specific to those sports.

In many sporting activities, protective eyewear is either required or recommended by a sport's governing body. US Lacrosse, the US Squash Racquets Association, and the USA Racquetball Association, as well as many state high school athletic associations, mandate the use of protective eyewear during play.

Many groups such as the Prevent Blindness America, the Coalition to Prevent Sports Eye Injuries, and other sport governing bodies and medical organizations recommend eye protection for any sport or recreational activity where a ball or implement (e.g., stick or racquet) is used. It is estimated that there are 40,000 sports related eye injuries treated during hospital emergency room per year. This number does not include those injuries not presenting to emergency rooms, bringing the estimated total to over 100,000 yearly eye injuries. It is also estimated that over 40% of the people sustaining the injuries are 16 years old and younger.

Use of eye protection is also becoming more of a concern to parents and sports enthusiasts. Due to an increased public awareness, many people now choose to protect their eyes and/or their children's eyes from various balls, racquets, sticks, and body parts that may contact the eye during play.

Eye protectors have been developed by both sporting goods manufacturers and by ophthalmic and medically based companies, each having an interest in the protection of the eye and the reduction in the number and severity of injuries that occur during sports and recreational activities.

Eye protectors are also used in industrial settings where small projectiles such as metallic fragments or wood chips can enter the eye. Eye protection may provide protection from harmful ultra violet or other electromagnetic radiation that may damage the eye. Eye protectors may also protect the eye from wind and dirt or any particle that may come in contact with the eye including liquids, contaminants, and microbial debris.

Many people play multiple sports and would require certified eye protection for more than one sport, it's balls and/or implements. Having a single pair of protective eyewear that passes testing requirements for all sports is simple, but it does not allow for unique, sports specific designs, and does not afford the wearer the best view during use. Having many different pairs of eye protectors would be cumbersome and expensive.

In lacrosse and field hockey, balls and sticks are used in both sports. Other sports that use balls and sticks or where finger or body contact could occur are squash, racquetball, paddleball, basketball, baseball, badminton, handball, soccer, and tennis, for example. Injury data has been collected and analyzed by several groups of researchers. Although severe injuries are rare, there have been broken facial bones specifically to the eyes and nose along with injuries to softer tissues such as the eyes. Protection would likely prevent a catastrophic injury from occurring. There are also many other sports in which eye injuries can occur including all racquet sports, baseball, basketball, and paint ball to name a few. Eye protectors are designed to minimize or significantly reduce injury to the eye. Eye protectors do not eliminate the possibility of an eye injury as playing sports with balls, sticks, racquets, or where fingers and/or body parts may contact the eye, is inherently dangerous.

Some problems inherent to eyewear with lenses used during sports, in particular outdoor sports, are scratching, fogging, and rain on the lens, all decreasing visibility for the player. Selecting a lens material onto which a scratch resistant coating can be applied can reduce scratched lenses. Also, providing each player with a soft pouch or case for storage would be helpful. Fogging can be reduced by proper airflow and ventilation through the frame and also by moving the lenses and frame away from the face. Additionally, an anti-fog coating, either factory applied or supplied as a wipe that the user can apply, can be used to reduce fogging and condensation on the lens. Visibility issues caused by rain hitting the lenses can be improved by applying a water repellant coating or wiper device to the outside surface.

Lenses and lens material, by nature, have optical properties. Certain impact resistant materials when formed in the shape of a “wrap” around the head design and thereby having a certain radius of curvature induce aberrations in the lens such as induced prism and spherical aberration. These aberrations distort vision when looking through the lens. Adjustments of the optics of the lenses as well as physical design changes can reduce this distortion.

Thus there is a need for a sports eye protector that overcomes the above mentioned and other disadvantages.

### SUMMARY OF INVENTION

In one aspect, there is a sports eye protector that includes a frame, a padding in communication with the frame and a sports grille releasably attachable to the frame. In another aspect, there is a sports eye protector that includes a frame and a sports grille releasably attachable to the frame, wherein the sports grille is compliant with a testing standard associated with a sport. In yet another aspect, there is a sports eye protector that includes a frame configured to be releasably attachable to any one of a plurality of sports grilles, wherein the plurality includes a first sports grille associated with a first sport and a second sports grille associated with a second sport.

Any of the above aspects can include one or more of the following features. The sports grille can include a single vertical element, crossed elements in a T shape, crossed elements in a V shape, or any combination thereof. The sports grille can include a configuration of elements that prevent a specific object from impacting an eye of the wearer. The specific object can be a ball associated with a sport, a stick associated with a sport, a racquet associated with a sport, or any combination thereof. The sports grille can include an impact lens.

The sports grille can be configured so that it passes a sport specific testing requirement. The sports grille can be compliant with a testing standard associated with a sport. The testing standard can be associated with American Society for Testing and Materials (ASTM) F803.

The padding can include a dense foam. The sports eye protector can include a head strap in communication with the frame. The head strap can include a split strap. The frame can have a shape that contours around a curvature of a face of a wearer. The frame can include a clip receptacle.

The sports can include, without limitation, lacrosse, field hockey, squash, racquetball, paddleball, basketball, baseball softball, badminton, handball, soccer, and/or tennis.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is an exploded view of the disclosed sports eye protector;

FIG. 2 is a front view of one embodiment of a sports grille;

FIG. 3 is a front view of another embodiment of a sports grille; and

FIG. 4 is a front view of still another embodiment of a sports grille.

### DETAILED DESCRIPTION

This description relates to a sports eye protector, and more specifically, describes a preferred embodiment of a sports eye protector that allows the wearer to play multiple sports with one frame. Different embodiments of a sports grille, an interchangeable component that releasably couples to the frame, allows the wearer to play different sports using the same frame. Each sports grille is specific for a sport(s) and/or a sport's testing standard, such as ASTM F803. A sports grille can include a well-ventilated design with many open-air portions comprising one to several design elements. This allows better ventilation and less or no fogging, improved visibility during rainy or misty conditions encountered in outdoor sports. There may be multiple embodiments of a sports grille. A single eye protector may pass testing requirements for a single sport. This invention enables the wearer to play multiple sports, and provides to the wearer a sport specific design that maximizes vision, allows superior venting, all while taking into consideration the balls or pucks, sticks, or body parts encountered for each sport.

Eye protection, as it is described herein, is protection of the eye, as defined in, but not exclusively to ASTM F803-03 for example, from balls, sticks, racquets, and/or body parts.

Having an eye protector, with an interchangeable sports grille, for example, that is adaptable to different sports can be designed for specific sports, taking into consideration, balls and implements, finger or body contact, field of view, fogging and visibility issues unique to that sport. The eye protector can perform a single function of protection during sports and each sports grille configuration can be sport specific.

Applicant is aware of no sports eye protector that has a sport specific interchangeable sports grille that releasably couples to a frame, and allows the wearer to play multiple sports by interchanging those sport specific sports grilles. In the described embodiments, any single embodiment of an interchangeable sports grille is specific for each sport and each sport's ASTM F803 testing requirements, for example. This allows the wearer to play multiple sports by interchanging a sports grille depending on the sport being played. Embodiments of the sports grille are specific to the balls, projectiles, pucks, sticks, racquets, or implements, and the various body parts that have been identified in and that are tested for in a guideline or standard. Additionally, the sports grille is designed bearing in mind the environment the sport is played in, indoors or outdoors, and how those environments can affect visibility of the user.

A device that reduces the aforementioned problems inherent to impact resistant lenses, namely scratching, fogging, and distorted optics, is an eye protector device that consists of a frame and a releasably coupled sports grille, that still complies with the eye protection standards, such as ASTM 803 for various sports. A sports grille, can be made of any material, or combination of materials, that when releasably coupled to the frame, passes the sport specific testing requirements. The grille could be configured with any number of elements such as bars, lenses and bar/lens combinations, screens, webs, or interwoven patterns of elements for example. These configurations will dampen the blow of the ball, sticks, racquets or fingers. A configuration could be specific to an ASTM F803 testing requirement, for example. Any reduction in total useable visual fields would be as minimal as possible in the design to allow the wearer the greatest use of their field of view, of each eye.

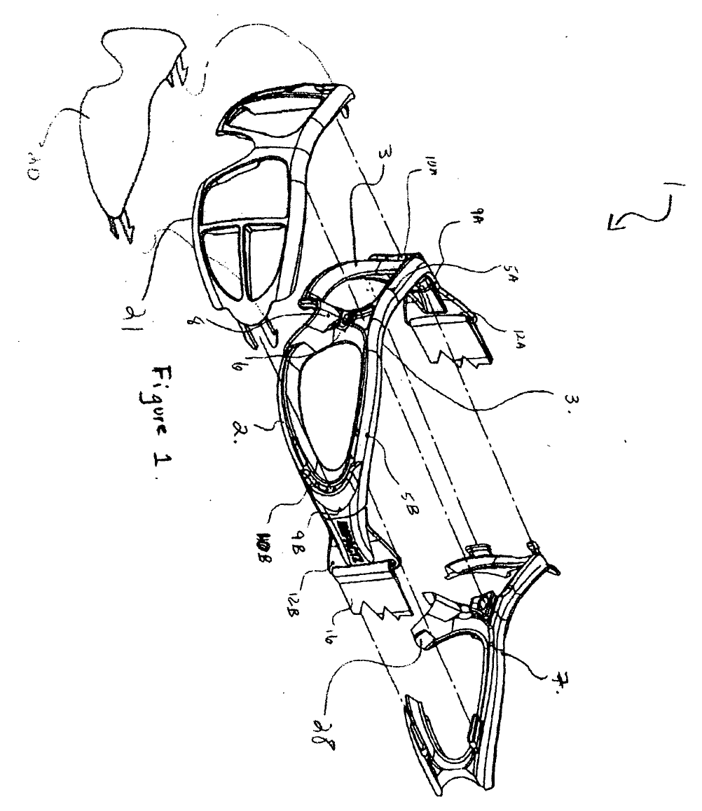

FIG. 1 is an exploded view of the disclosed sports eye protector 1. The sports eye protector 1 comprises a frame 2, a sports grille 21, and cushion 7. The frame 2 may be constructed of an impact resistant material, such as but not limited to polycarbonate. The frame 2 may be transparent, semi-transparent, a single opaque color, or multicolored, with or without logos or emblems. The frame 2 comprises a Frame Front 3, a Right Eye Piece 5a, a left eye piece 5b, a bridge 6, a nose guard 8, right side piece 9a, left side piece 9b, right lens clip receptacle 10a, and left lens clip receptacle 10b. The frame 2 may have a shape that “wraps” or contours around the curvature of the face of a wearer. The center of the frame 2 is the bridge 6. The frame 2 may bend symmetrically from the bridge 6 around the face over the eyes, toward each ear of a wearer, and terminates near the temples of the wearer. The frame 2 may come in various predefined sizes to accommodate different size heads. For example, two predefined sizes can be a size to accommodate a statistically average female adult and a second size to accommodate a statistically average male adult. The design takes into consideration testing requirements, fit and optical issues, comfort, style and weight issues. The design possibilities are limitless, depending on the imagination of the designer, and are encompassed by this patent application.

In one disclosed embodiment, the frame 2 may have a feature where a sports grille 21 can be inserted into the frame. The sports grille 21, may releasably couple to the frame 2. When coupled to the frame 2, the sports grille 21 is secure enough to absorb impact from balls of various sports and/or prevent penetrating injury from implements or body parts. Many sports grille 21 configurations can be designed as balls, sticks, racquets, and testing requirements vary from sport to sport. This makes it more likely that a ball and or stick or racquet cannot pass through the frame 2 and sports grille 21 combination and contact the eyes of the wearer. This is advantageous, as the user can interchange sports grilles 21 for different sports. The user can also interchange the sports grille 21 with a sports grille 20 that includes a lens. The sports grilles 21 are sports specific and because they releasably couple to the frame 2, they can be easily interchanged for use in multiple sports.

The sports grille 21, when used, can be made of any material or combination of materials that absorb the blow of the ball or sticks or racquets. The sports grille 21 is configured to fit into terminal end clips which are inserted into the lens clip receptacles 10a, 10b. The sports grille 21 spans over the bridge 6 and nose guard 8 of the frame front 3. The sports grille 21 may be formed of any impact resistant materials, such as but not limited to polycarbonate. The design of the sports grille may reflect the sport or sports it is to be used in.

The Lens sports grille 20, when used, can be formed from any impact resistant, UV absorbing, transparent material such as, but not limited to polycarbonate. The Lens 20 is configured to fit into terminal lens clips located in the lens clip receptacles 10a, 10b. The lens 20 spans over the bridge 6 and Nose guard 8 of the frame front 3. The front and back curvatures of the Lens 20 are dictated by the curvature of the Frame 2. The shape of the lens 20 is limitless, depending on the imagination of the designer, and such shapes are encompassed in this patent application and include shapes such as oval shaped or trapezoidal shaped. Lens design and shape will also take into consideration maximizing peripheral vision and minimizing distortion of the wearer, and at the same time contouring to the wearer's face. Coatings may be applied to the lenses including but not limited to scratch-resistant, fog resistant, anti-glare, hydrophilic, and hydrophobic.

The cushion 7 comprises a nose pad 28. The nose pad 28 is contiguous with the cushion 7 that lines the inner surface (the side that faces the user's face) of the frame 2. The Nose Pad 7 may be made of any elastomer material, including but not limited to: santoprene. The elastomer material shall conform to the wearer's nose contours when on and be as non-slip as possible, having protrusions or finger-like projections. The color may be transparent, semi-transparent or an opaque hue and the nose pad can be of various shapes and sizes. The nose pad 7 serves as a resting point for the frame upon the wearer's nose and assists in holding the frame 2 in place when on a wearer's head.

The Nose Guard 8 may be made of any impact resistant material including, but not limited to: metal, plastic polymer, polycarbonate. The nose guard 8 may originate on right Eye Piece SA, span above the nose and end on left Eye Piece 5B. The nose guard 8 may be a continuous part of the frame if molded as one piece with the frame or a separate piece or pieces secured by an adhesive, for example. The nose guard 8 may be of the shape and size to accommodate most nose sizes and shapes. It covers and protects the nasal bones of the wearer when on the wearer's head.

Side Pieces 9A and 9B may be made of an impact resistant material, such as, but not limited to polycarbonate. Right Side Piece 9A is continuous with right Eye Piece SA and let Side Piece 9B is continuous with left Eye Piece 5B. All are part of the frame 2. Side Pieces 9A, 9B, are disposed approximately parallel to the sides of the head and located near either temple. The Side Pieces 9A and 9B, terminate in a right Strap Clip Receptacle 10A and a Left strap clip receptacle 10B. The Strap Clip Receptacles 10A, 10B may be designed in various ways for the insertion of right Strap Clip 12a and left strap clip 12b. The Side Pieces 9A, 9B also have Lens Clip Receptacles 10a, 10b to accommodate an impact lens 20 when needed.

The right and left Strap Clips Right 10A, 10B may be continuous with right and left Side Pieces 9a, 9b, and molded as one piece, or separate pieces, and have an opening for a Head Strap.

16. The Head Strap 16 then rests in a strap slot. An element known as a strap stop is the element that prevents the strap from pulling out when worn by a user. Right and left Strap Clips 12a, 12b are attached to the frame 2. The attachment means may be, but not limited to: a pin or screw.

Alternatively, the Head Strap 16 may be attached to the frame 2 by a number of additional ways such as, but not limited to: a snap, clasp, or other like devices that allow for a quick and simple engagement and disengagement.

In one embodiment, the head strap 16 may be about ¾″ to about 1″ wide. If the head strap is a Split Strap, the straps may be about 1/16″ to about ⅛″ wide. The length of the head strap 16 may be adjustable and vary from a length of about 6″ to about 18″. The length of the head strap 16 can be associated with the predefined frame sizes. The head strap 16 may, in a first embodiment have an elasticized portion and a non-elasticized, and in a second embodiment may be elasticized its entire length. In the first embodiment, the elasticized portion is around the head while the non-elasticized portion is at its connection to the frame 2. The portion connecting to the Strap Clip 12a or 12b may be in the shape of a closed loop. The head strap 16 may have a buckle, that allows adjustment for various head sizes. The head strap 16 can be of many colors or designs, and could have non-slip features or a softer lining for more comfort.

The cushion 7 which sits between the frame 2 and the wearer's face may be made of any suitable material that provides cushioning, such as, but not limited to: dense closed cell foam padding. The cushion 7 will contour along the frame 2 and be positioned in various places along the frame's inner edge. The cushion 7 may be about ¼″ thick, for example, and of various widths and lengths depending on the location. The material comprising the cushion 7 will be hypoallergenic. The cushion 7 can be fastened to the frame 2 by an adhesive or the foam can be directly injection molded into the frame. The cushion 7 can also be simply pressed through the frame 2 by nibs or protruding tabs and removed by doing the reverse or pulling the nibs away from the Frame 2. This cushion 7 is absorbent with respect to the impact of an object striking the eyewear. The cushion 7 is strategically place along the inner edge of the frame 2 to absorb impact in certain areas while diverting impact from others, such as the eye and surrounding structures.

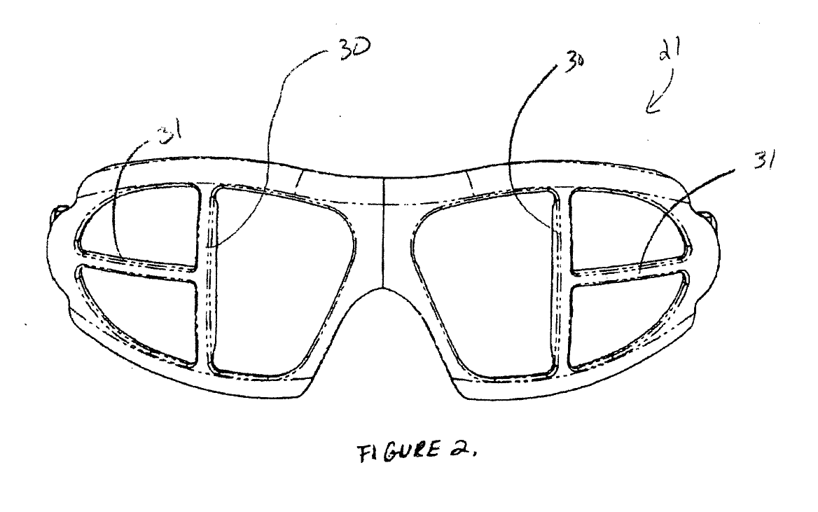

FIG. 2 is a front view of a preferred embodiment of a sports grille. The configuration of the materials and the material choices are limited only to the imagination of the designer, allowing a multitude of different sports grilles for a variety of sports. Sports grilles 21 are interchangeable with one another, providing the user with protection for a number of different sports. The sports grille 21 shown in FIG. 2 has a “T-shape” as shown by the crossed elements 30 and 31. Each element 30,31 is made as thin as possible to not affect the field of vision, each element placed strategically as possible not to affect the field of vision. This design is to stop a lacrosse ball at 45 MPH for example.

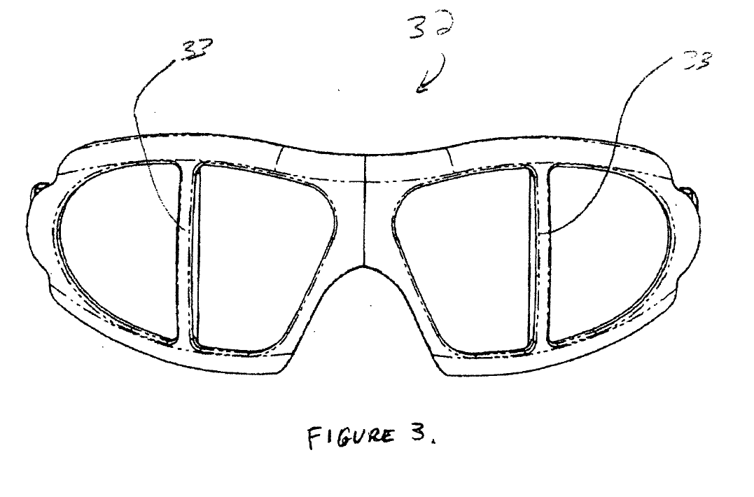

FIG. 3 is a front view of another embodiment of a sports grille 32. This embodiment has a single vertical element 33 in the field of view of each eye. Each element made as thin as possible to not affect the field of vision, each element placed strategically as possible not to affect the field of vision. This embodiment is designed for field hockey, to stop a ball at 55 MPH, and the stick penetration test, for example.

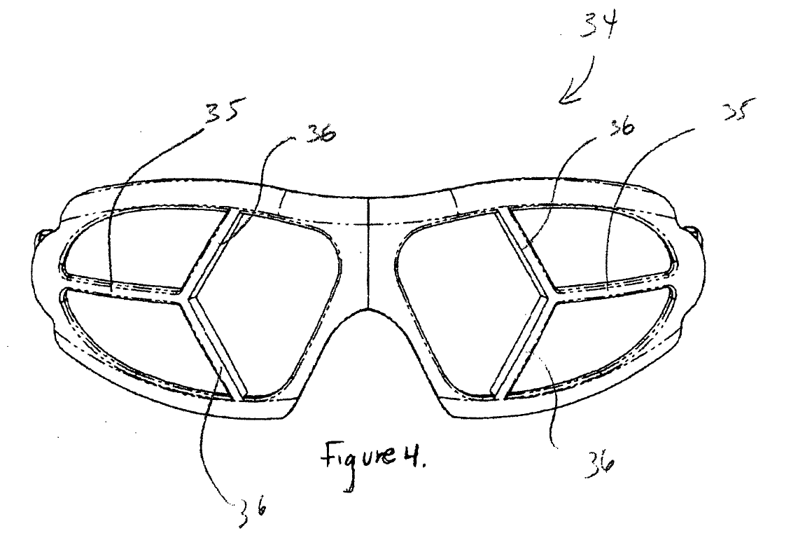

FIG. 4 is a front view of another embodiment of a sports grille 34. This embodiment has a single horizontal element 35 and a single V-shaped element 36 spanning top to bottom in the field of view of each eye. Each element made as thin as possible to not affect the field of vision, each element placed strategically as possible not to affect the field of vision. This embodiment is for women's lacrosse at 60 MPH.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but include all embodiments falling within the scope of the appended claims.

What is claimed is:

1. A sports eye protector comprising:
a frame;
a padding in communication with the frame; and
a sports grille releasably attachable to the frame.

2. The sports eye protector of claim 1, wherein the sports grille comprises a single vertical element.

3. The sports eye protector of claim 1, wherein the sports grille comprises crossed elements in a T shape.

4. The sports eye protector of claim 1, wherein the sports grille comprises crossed elements in a V shape.

5. The sports eye protector of claim 1, wherein the sports grille comprises a configuration of open-air elements that prevent a specific object from impacting an eye of the wearer.

6. The sports eye protector of claim 5, wherein the specific object is a ball associated with a sport, a stick associated with a sport, a racquet associated with a sport, or any combination thereof.

7. The sports eye protector of claim 1, wherein the sports grille comprises an impact lens.

8. The sports eye protector of claim 1, wherein the padding comprises a dense foam.

9. The sports eye protector of claim 1, wherein the sports grille passes a sport specific testing requirement.

10. The sports eye protector of claim 1, wherein the sports grille is compliant with a testing standard associated with a sport.

11. The sports eye protector of claim 1, wherein the testing standard is associated with American Society for Testing and Materials (ASTM) F803.

12. The sports eye protector of claim 1, further comprising a head strap in communication with the frame.

13. The sports eye protector of claim 1, wherein the head strap comprises a split strap.

14. The sports eye protector of claim 13, wherein the frame has a shape that contours around a curvature of a face of a wearer.

15. The sports eye protector of claim 1, wherein the frame includes a clip receptacle.

16. A sports eye protector comprising:
a frame; and
a sports grille releasably attachable to the frame, the sports grille being compliant with a testing standard associated with a sport.

17. The sports eye protector of claim 16, wherein the testing standard is associated with American Society for Testing and Materials (ASTM) F803.

18. The sports eye protector of claim 16, wherein the sport is lacrosse, field hockey, or both.

19. A sports eye protector comprising:
a frame configured to be releasably attachable to any one of a plurality of sports grilles, the plurality including a first sports grille associated with a first sport and a second sports grille associated with a second sport.

20. The sports eye protector of claim 19, wherein the first sport is lacrosse and the second sport is field hockey.
